(19) United States
(12) Patent Application Publication
(10) Pub. No.: US 2024/0008735 A1
(43) Pub. Date: Jan. 11, 2024

(54) COAXIAL MULTI-ILLUMINATED OCULAR IMAGING APPARATUS

(71) Applicant: TRANSFOLIO, LLC, Irving, TX (US)

(72) Inventors: Henricus Mathias Marie KESSELS, Weert (NL); Emmanuel Leopold A. SNEYDERS, Beerse (BE); Lieven Maria K. CAUWENBERGHS, Beerse (BE); Jeroen SNEPVANGERS, Van Nuys, CA (US)

(21) Appl. No.: 18/035,036

(22) PCT Filed: Nov. 2, 2020
(86) PCT No.: PCT/US2020/058575

§ 371 Date: May 2, 2023

(51) Int. Cl. A61B 3/00; A61B 3/18; A61B 3/14

(52) U.S. Cl. CPC A61B 3/0008; A61B 3/18; A61B 3/14; A61B 3/1216

(57) ABSTRACT

Methods and systems for imaging a subject's eyes are provided. In one embodiment, an optical imaging apparatus is provided that includes a plurality of eye imaging systems. Each eye imaging system may include a camera sensor with a viewing direction oriented towards a subject whose eyes are imaged. The eye imaging systems may also include at least one coaxial light source, which may be configured to provide light coaxially aligned with the viewing direction of the camera sensor. The eye imaging systems may further include supplementary light sources configured to light an eye from different angles.

### FIELD OF THE INVENTION

The invention relates generally to methods and apparatuses for ocular imaging, and more particularly to a method and apparatus for measuring the shape and colors of the human eye, of which the sclera, iris and pupil are captured specifically. Such images may be for the purpose of rendering a life-like three-dimensional digital eyes for general use in all areas of computer graphics imaging (CGI), including visual effects in film and image production, real-time computer gaming, and all forms of virtual reality and artificial intelligence avatars.

### BACKGROUND

Making three-dimensional digital characters in CGI life-like that render realistically from all angles and in all lighting situations requires both an accurate understanding of the shape and micro-structures well as an accurate understanding of the colors, both real and structural, under all environmental circumstances. The shape of objects in CGI may be typically stored as a three-dimensional mesh of polygons. The polygons are a linear approximation of the shape of the actual object. In general, the more unique polygons are used, the higher the accuracy of the approximation. Shape stored as polygons may be referred to as geometry or mesh. Creating accurate versions of such geometry or mesh can require scanning of real-world versions of the objects to be replicated in CGI.

### SUMMARY

The present disclosure presents new and innovative systems and methods for imaging a subject's eyes. In a first aspect, an optical imaging apparatus is provided comprising a plurality of eye imaging systems. Each eye imaging system comprises a camera sensor with a viewing direction oriented towards a subject whose eyes are imaged and at least one coaxial light source. The coaxial light source may be configured to provide light coaxially aligned with the viewing direction of the camera sensor. Each eye imaging may also comprise a plurality of supplementary light sources configured to light an eye from different angles.

In a second aspect according to the first aspect, the at least one coaxial light source includes a cross light to provide a fixation target for the subject during imaging.

In a third aspect according to the second aspect, the cross light is oriented perpendicular to the viewing direction of the camera sensor.

In a fourth aspect according to the third aspect, light from the cross light is redirected along the viewing direction by a beam splitter.

In a fifth aspect according to any of the first through fourth aspects, the at least one coaxial light source includes a center light to illuminate the eye during imaging.

In a sixth aspect according to any of the first through fifth aspects, light from the center light is redirected along the viewing direction through a mirror box including at least one mirror and at least one beam splitter.

In a seventh aspect according to any of the first through sixth aspects, the at least one coaxial light source includes a cross light and a center light.

In an eighth aspect according to any of the first through seventh aspects, the plurality of supplementary light sources includes at least two of: (i) temporal lights angled distally away from a centerline of the eye, (ii) nasal lights angled proximally away from a centerline of the eye, (iii) top lights angled above the eye, and (iv) bottom lights angled below the eye.

In a ninth aspect according to any of the first through eighth aspects, the optical imaging apparatus is configured to take a plurality of images using at least a subset of the plurality of supplementary light sources. At least a subset of the plurality of images may be captured by illuminating a single supplementary light source from among the subset of the plurality of supplementary light sources.

In a tenth aspect according to any of the first through ninth aspects, the optical imaging apparatus can adjust and measure a light intensity of the at least one coaxial light source and the plurality of supplementary light sources.

In an eleventh aspect according to any of the first through tenth aspects, the at least one coaxial light source and the plurality of supplementary light sources can illuminate on a continuous basis and a strobe basis.

In a twelfth aspect according to any of the first through eleventh aspects, the optical imaging apparatus can adjust and measure an angle of the supplementary light sources.

In a thirteenth aspect according to any of the first through twelfth aspects, the optical imaging apparatus further includes a chin rest and forehead restraint. The optical imaging apparatus may be able to adjust and measure at least one of (i) a distance between the plurality of eye imaging systems and the chin rest and forehead restraint and (ii) a distance between the at least one coaxial light source and the chin rest and forehead restraint.

In a fourteenth aspect according to any of the first through thirteenth aspects, the at least one coaxial light source and the plurality of supplementary light sources are configured to emit light in at least a portion of the visual electromagnetic spectrum.

In a fifteenth aspect according to the fourteenth aspect, the at least one coaxial light source and the plurality of supplementary light sources are configured to emit light at least partially in the near infrared light spectrum.

In a sixteenth aspect according to any of the fourteenth and fifteenth aspects, the camera sensor is configured to detect light in at least the portion of the visual electromagnetic spectrum emitted by the at least one coaxial light source and the plurality of supplementary light sources.

In a seventeenth aspect according to any of the first through sixteenth aspects, the optical imaging apparatus is configured to vertically adjust a position of the eye imaging systems and to horizontally adjust a position of the eye imaging systems.

In an eighteenth aspect, an eye imaging system is provided comprising a camera sensor with a viewing direction oriented towards a subject whose eyes are imaged and at least one coaxial light source, wherein the coaxial light source is configured to provide light coaxially aligned with the viewing direction of the camera sensor. The eye imaging system may also include a plurality of supplementary light sources configured to light an eye from different angles.

In a nineteenth aspect according to the eighteenth aspect, the at least one coaxial light source includes a cross light to provide a fixation target for the subject during imaging.

In a twentieth aspect according to the nineteenth aspect, the cross light is oriented perpendicular to the viewing direction of the camera sensor.

The features and advantages described herein are not all-inclusive and, in particular, many additional features and advantages will be apparent to one of ordinary skill in the art in view of the figures and description. Moreover, it should be noted that the language used in the specification has been principally selected for readability and instructional purposes, and not to limit the scope of the disclosed subject matter.

### BRIEF DESCRIPTION OF THE FIGURES

FIGS. 1A-B depict a top views of an optical apparatus according to an exemplary embodiment of the present disclosure.

FIGS. 2A-D depict a left three-quarter view of an optical apparatus according to an exemplary embodiment of the present disclosure.

FIGS. 3A-E depict a left three-quarter view of an optical apparatus according to an exemplary embodiment of the present disclosure.

FIGS. 4A-B depict a left view of an optical apparatus according to an exemplary embodiment of the present disclosure.

FIGS. 5A-B depict a rear view of an optical apparatus according to an exemplary embodiment of the present disclosure.

FIGS. 6A-B depict a front view of an optical apparatus according to an exemplary embodiment of the present disclosure.

FIGS. 7A-B depict a front three-quarter view of an optical apparatus according to an exemplary embodiment of the present disclosure.

FIGS. 8A-D depict a front three-quarter top view of an optical apparatus according to an exemplary embodiment of the present disclosure.

FIGS. 9A-D depict a top view of an optical apparatus according to an exemplary embodiment of the present disclosure.

FIGS. 10A-D depict a top view of an optical apparatus according to an exemplary embodiment of the present disclosure.

FIGS. 11A-B depict a right rear three-quarter view of an optical apparatus according to an exemplary embodiment of the present disclosure.

FIGS. 12A-E depict images captured using an optical apparatus according to an exemplary embodiment of the present disclosure.

FIGS. 13A-D depict lighting scenarios when imaging a subject's eye according to exemplary embodiments of the present disclosure.

### DETAILED DESCRIPTION OF EXAMPLE EMBODIMENTS

To accurately depict an object, it may be necessary to properly depict the microstructure of the surface of the object. Properly depicting the microstructure may require real-world imaging of the object. In particular, properly depicting an eye using CGI may require one or more images of the eye to be captured. These captures may be more accurate when incident light direction, incident light intensity, lens distortion, camera positioning, eye positioning, and/or head positioning can be controlled. Once controlled, images of the eye can be captured such that measurements can be made to interpret the size events and color events of the eye accurately. Size events may include capturing the refraction of the cornea as well as the micro shadow effects caused by the microstructure of the veins on the scleral surface and microstructure of the stroma of the iris. Color events may include both the wavelength absorption of melanin as well as various light scattering effects inside the cornea.

Existing optical imaging systems for capturing images of an eye typically use tripods, ring lights, and macro lenses. Tripods may stabilize the camera, but may fail to stabilize the head of the subject whose eyes are being captured. Tripods may also not suited for moving a few microns backward or forward to focus the optical system precisely on the iris and sclera. Ring lights may typically be positioned around the outside of the macro lens are illuminating the eye from a slight angle. The angle may change when the optical system is moved to focus and therefore the precise angle is often uncontrolled. Macro lenses distort the image of the eye, which can lead to measurement errors. In particular, macro lenses magnify objects at different distances differently, which causes unwanted parallax effects, occlusion of objects and inaccuracies in size measurements. Furthermore, such lenses may allow non-coaxial light (e.g., light that is not reflected directly from the eye towards the lens) to reach the sensor, creating additional light artifacts and noise that distort colors or obscure portions of the eye. Existing imaging systems typically do not use head restraints nor precise fixation points. Therefore, head and eye positioning are uncontrolled and will further distort color and size measurements. Such shortcomings may render existing imaging systems incapable of capturing images of the eye that are accurate enough to create CGI rendering of eyes with maximum realism.

One solution to this problem is to provide an ocular imaging system that includes a telecentric lens system aligned with the center of the eye to be imaged. Such a lens system does not rely on macro lenses to provide accurate focus, reducing the amount of non-coaxial light reflected from the eye that is captured by the lens and corresponding image. Such a device may include an optical system controlled on a footing that allows for 1 micron precise movements in x, left and right, y, up and down, and z, back and forth, directions. This may control the camera positioning and focusing. The subject whose eyes are being imaged is comfortably constraint in a chin and forehead restraint, which may limit head rotation and movements. Furthermore, such a device may include coaxial illumination may allow for greater control and illuminating the eye, as all light captured in an image originates from directly in front of the eye, reducing off-axis illumination unless desired. Furthermore, a second coaxial light source may be provided at a lower intensity for the subject of an image to look at while the image is captured. Such coaxial lights (optionally referred to as “fixation lights”) may provide greater accuracy of the image, as the subject's eyes have a target to focus on that is in the center of the lens. This ensures that the eye position is controlled and the iris plane is closer to perpendicular to the lens axis. Additional light sources at controlled light angles and intensity levels that are not coaxial may also be provided for further supplementary imaging, to provide comparison measurements of eye at other illumination angles. The combination of coaxial and non-coaxial illuminated images of the eye with controlled head and eye positioning, may provide a basis for various calculations regarding characteristics of the eye.

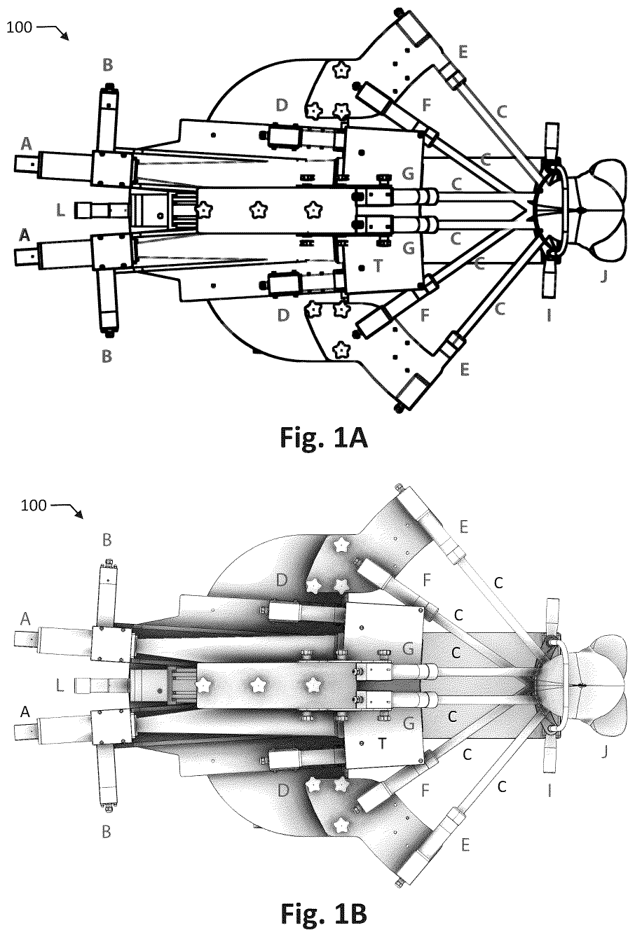

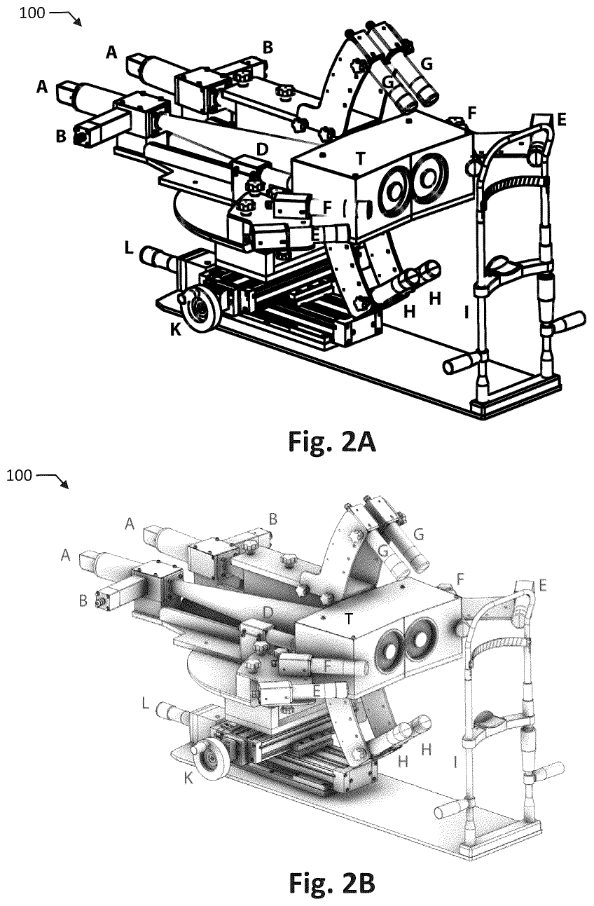

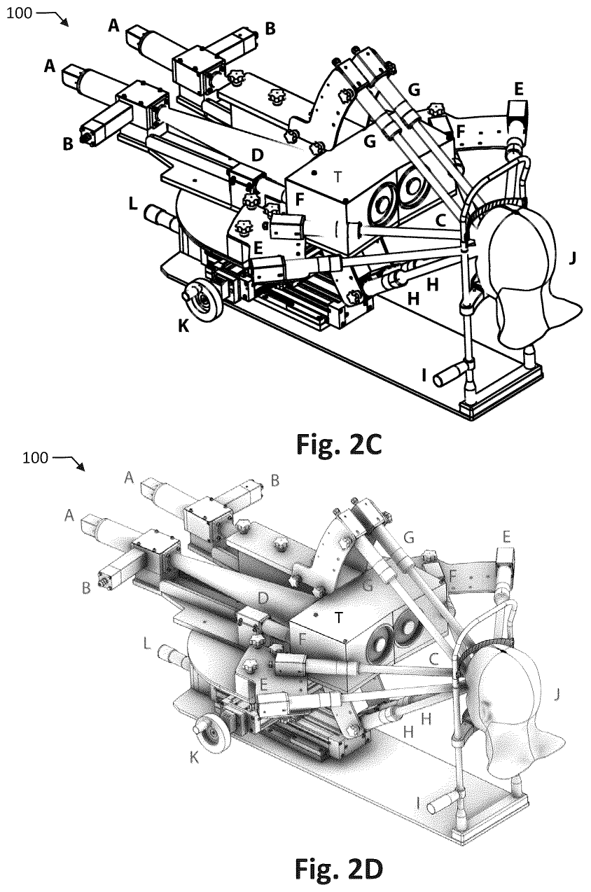

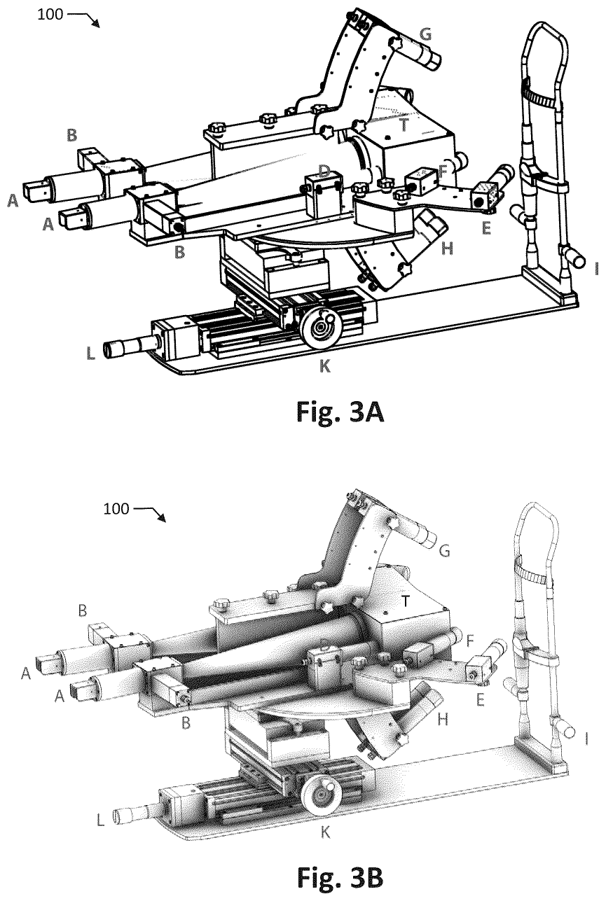

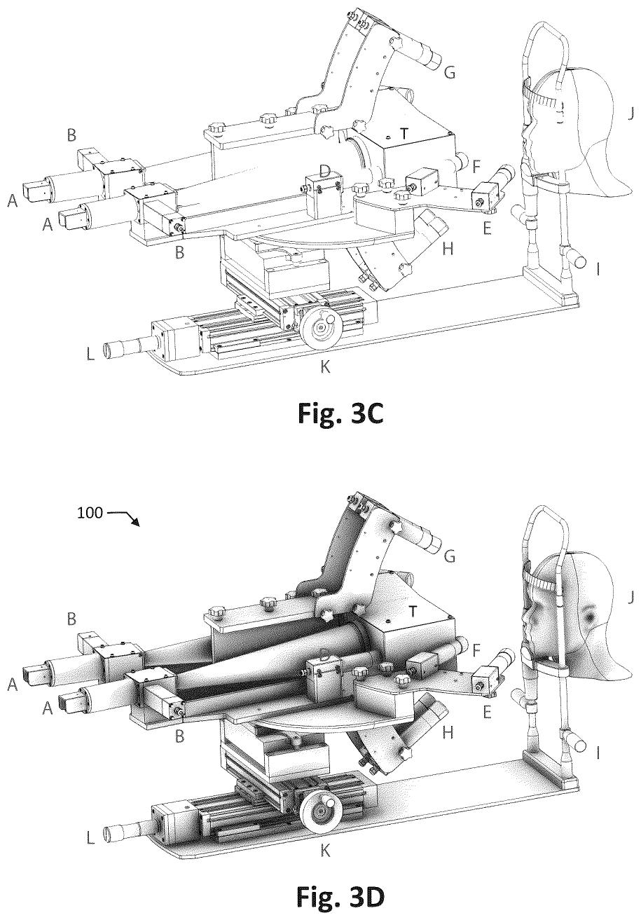

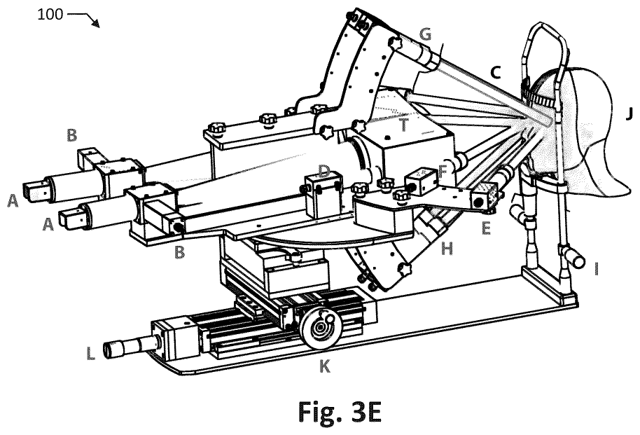

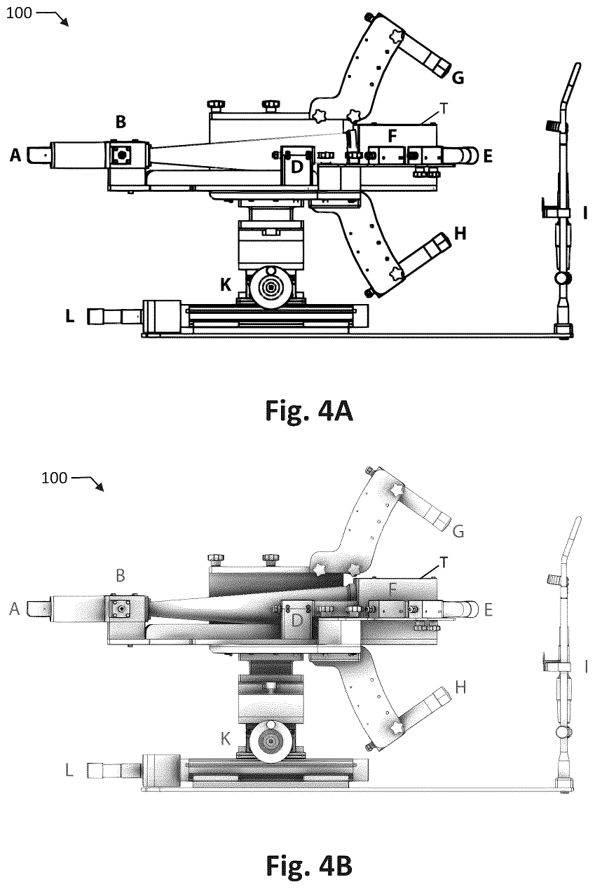

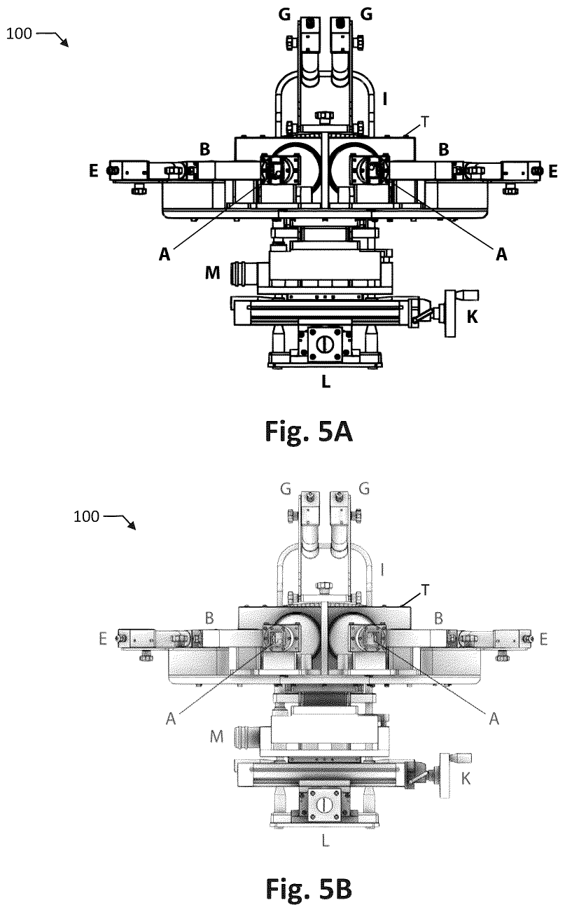

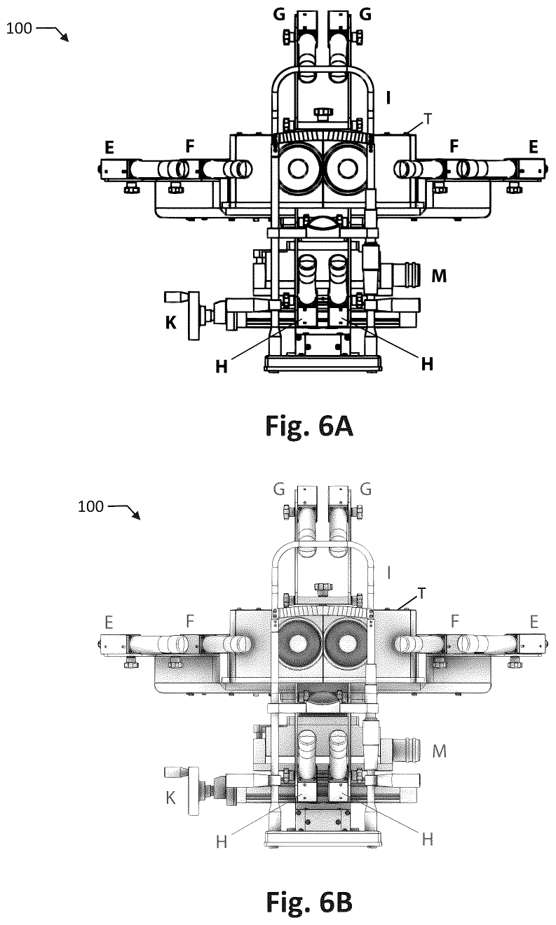

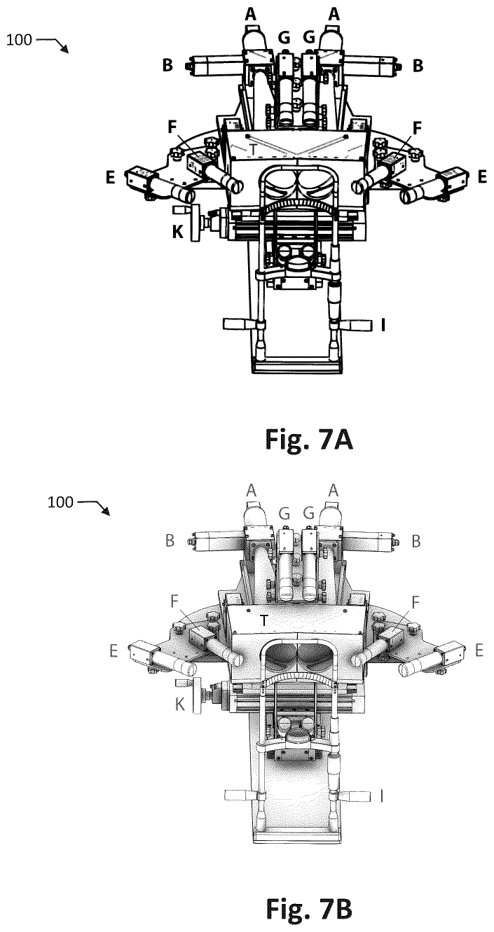

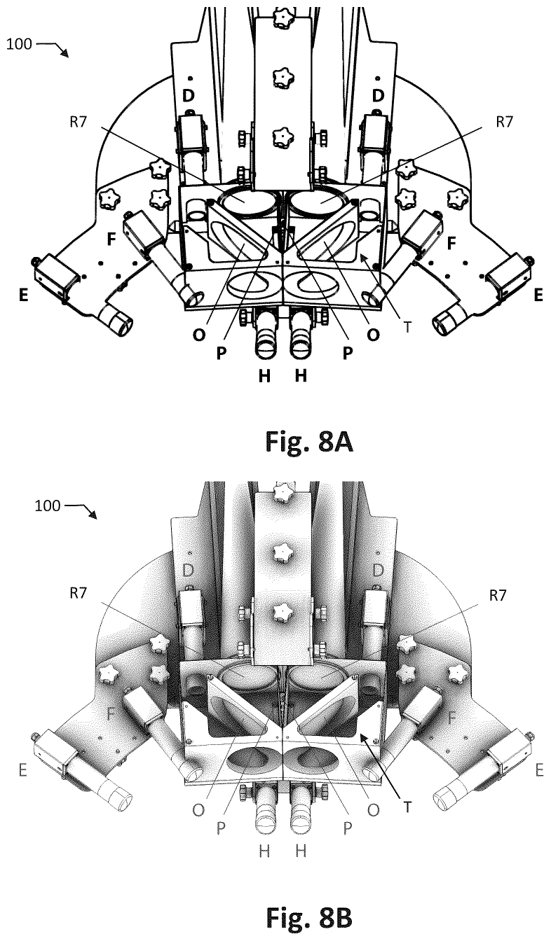

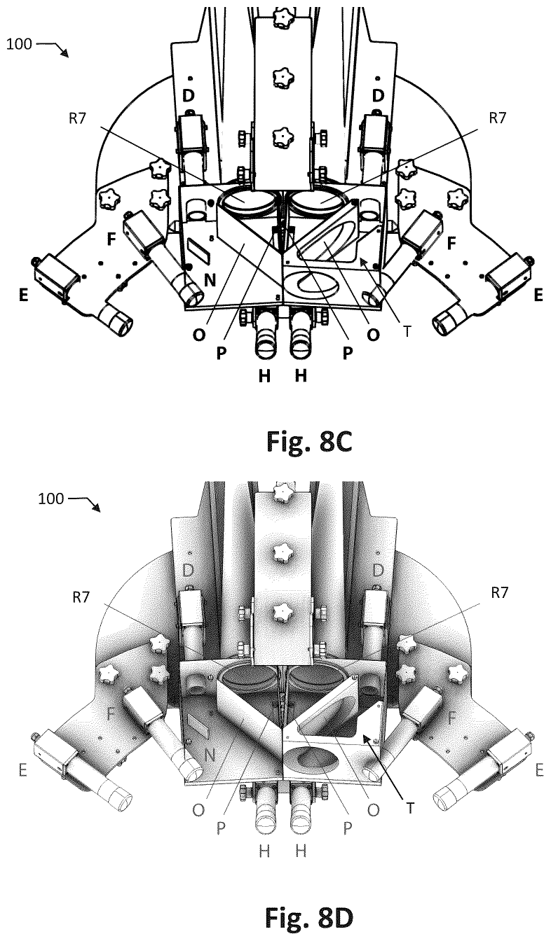

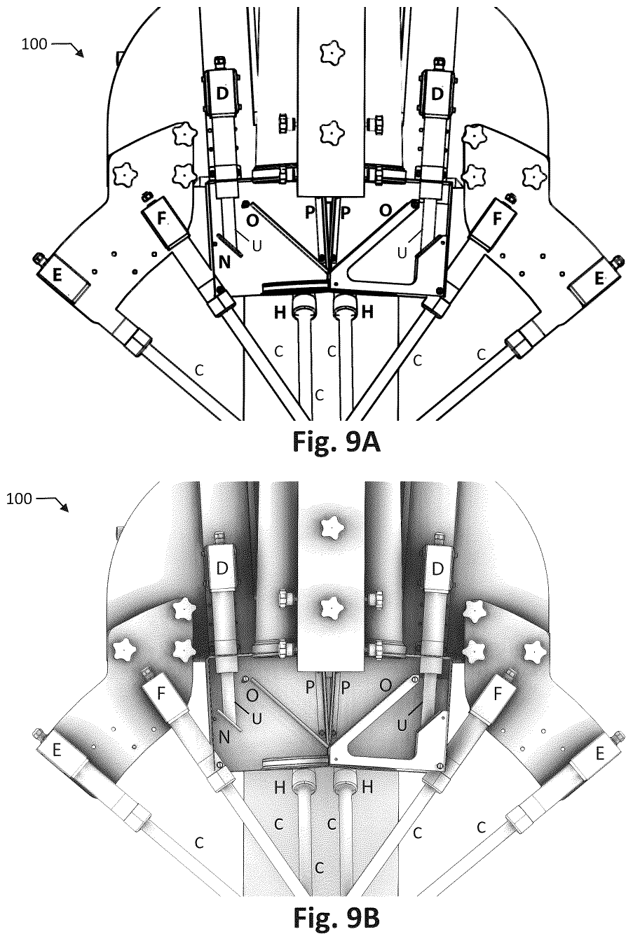

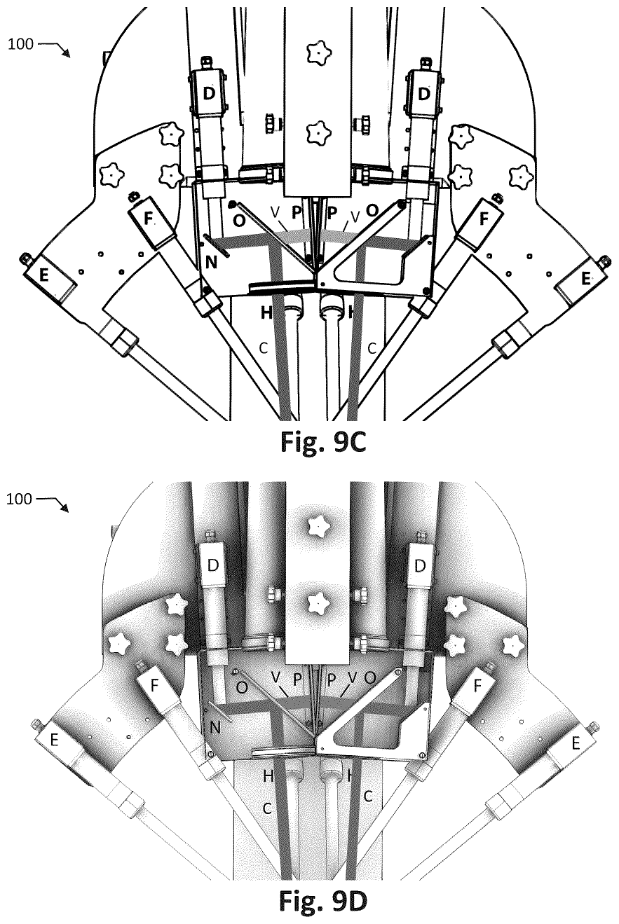

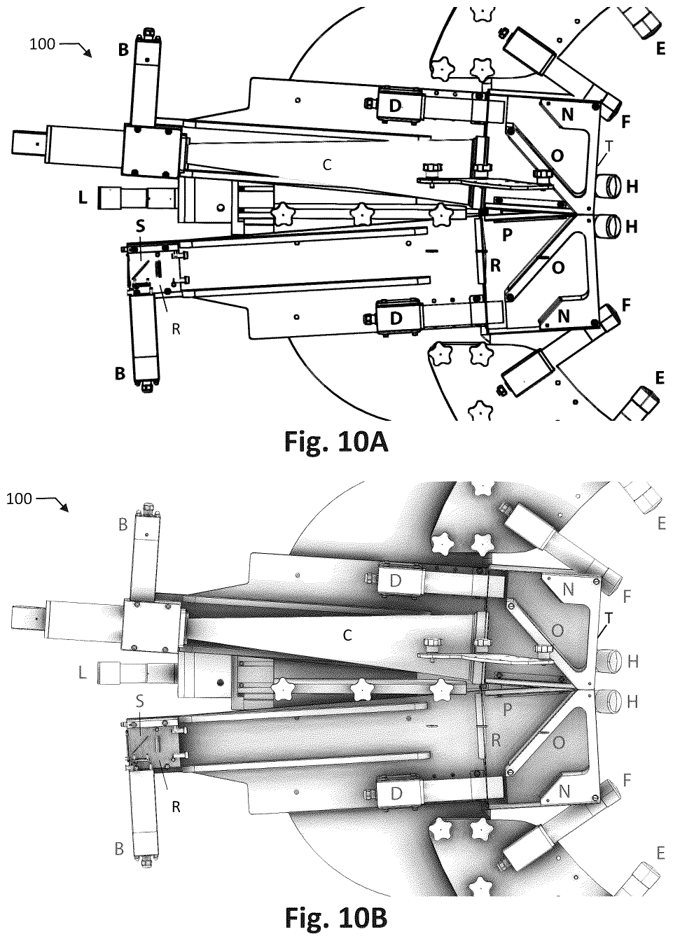

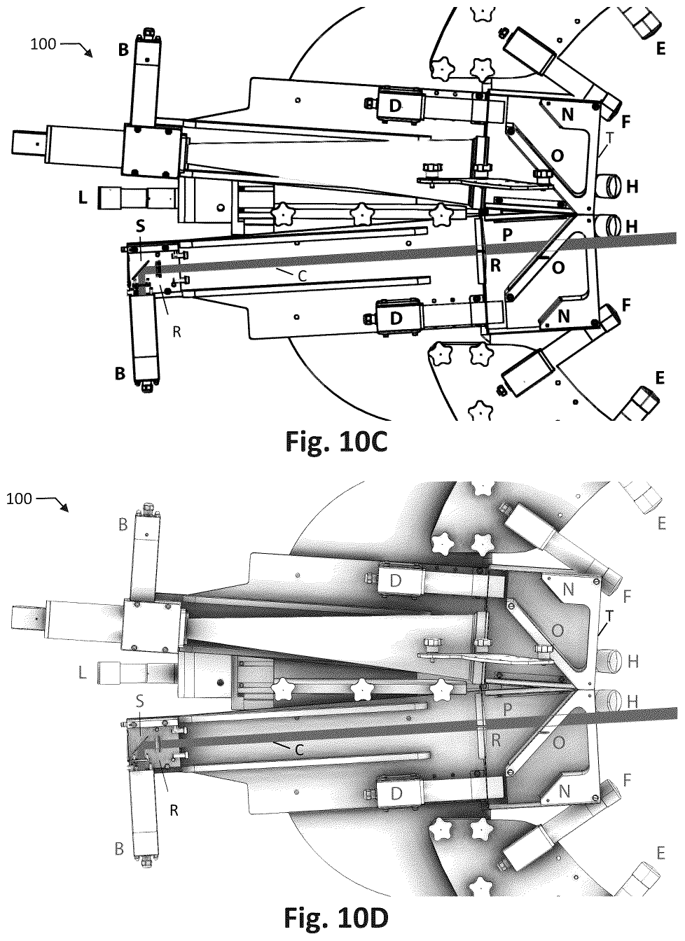

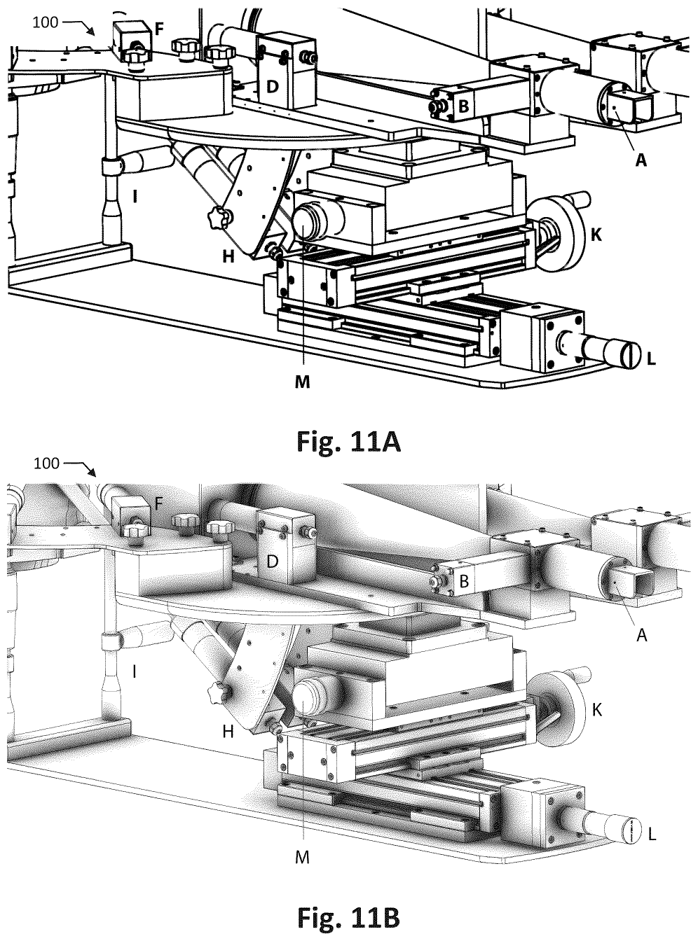

FIGS. 1-11 depict one such optical apparatus 100 from various views. In particular, FIGS. 1A-B depict a top view of the optical apparatus 100, FIGS. 2A-D depict a left three-quarter view of the optical apparatus 100, FIGS. 3A-E depict a left three-quarter view of the optical apparatus 100, FIGS. 4A-B depict a left view of the optical apparatus 100, FIGS. 5A-B depict a rear view of the optical apparatus, FIGS. 6A-B depict a front view of the optical apparatus 100, FIGS. 7A-B depict a front three-quarter view of the optical apparatus 100, FIGS. 8A-D depict a front three-quarter top view of the optical apparatus 100, FIGS. 9A-D depict a top view of the optical apparatus 100 with an interior view of a mirror box T, FIGS. 10A-D depict a top view of the optical apparatus 100, and FIGS. 11A-B depict a right rear three-quarter view of the optical apparatus 100.

The optical apparatus 100 includes two camera sensors A. A first camera sensor A may be positioned to image a left eye, alternatively referred to as the oculus sinister (OS). A second camera sensor A may be positioned to image a right eye, alternatively referred to as the oculus dexter (OD). The optical apparatus 100 may be symmetrical for each of the left eye and the right eye. For example, the optical apparatus 100 may include identical systems (e.g., identical lighting and imaging systems) for the left eye and the right eye. In certain implementations, such configurations may allow for both the left and right eyes of a subject (e.g., a person whose eyes are imaged) to be captured simultaneously. The optical apparatus 100 also includes a chin rest and forehead restraint I. The chin rest and forehead restraint I may be used to support the subject J while images are taken of the subject's J eyes. A height for the chin rest portion of the chin rest and forehead restraint I may be adjustable for improved comfort of the subject J and/or to properly position the subject's J eyes for imaging. Because the subject J is able to relax their support and facial muscles during the imaging, the chin rest and forehead restraint I may allow the eye to rest in its natural shape while being imaged. As can be seen in FIGS. 10A-D, the camera sensors A may include one or more lenses R.

The optical apparatus 100 may also be adjustable to ensure proper images of the subject's J eyes. The optical apparatus 100 may include various ways to adjust the positioning of the optical apparatus 100 relative to the subject J. For example, the position may be adjusted to ensure that one or more lights or camera sensors are properly aligned with the left or right eyes of the subject J. In certain implementations, the optical apparatus 100 may be adjustable in one or more of a lateral direction (e.g., left or right), a depth direction (e.g., forward or backward), and a vertical direction (e.g., up or down).

For example, FIGS. 11A-B depict various positioning systems for the optical apparatus 100. For example, the optical apparatus 100 may include a lateral positioning adjustor K, a depth positioning adjustor L, and a height positioning adjustor M. The lateral positioning adjustor K may be used to move the optical apparatus 100 left and right relative to the subject J. The height positioning adjustor M may be used to move the optical apparatus up and down relative to the subject J. The depth positioning adjustor L may be used to move the optical apparatus 100 forward or backward relative to the subject J. In particular, the depth positioning adjustor L may be used to adjust a focal length of the camera sensors A.

One or more of the lateral positioning adjustor K, the depth positioning adjustor L, and the height positioning adjustor M may be manually adjustable. For example, the lateral positioning adjustor K is depicted as including a crank handle. Rotating the crank handle may cause the optical apparatus 100 to move to the left or right. Additionally or alternatively, one or more of the lateral positioning adjustor K, the depth positioning adjustor L, and the height positioning adjustor M may be motorized. For example, as depicted the depth positioning adjustor L may be motorized, which may allow for greater control when focusing the cameras A. It should be understood that, although the figures depict a particular implementation of the optical apparatus 100, additional or alternative implementations may also be utilized. For example, although the lateral positioning adjustor K is depicted with a crank and handle for adjusting the lateral positioning of the optical apparatus 100, additional or alternative implementations may include a motorized lateral positioning adjustor.

Various lighting configurations may be used to illuminate the eyes of a subject J for imaging. Accordingly, the optical apparatus 100 may include multiple lighting devices. For example, the optical apparatus 100 includes cross lights B. The optical apparatus 100 also includes center lights D. The center lights D and/or the cross lights B may be configured to provide light that is coaxial to the subject's J eyes. For example, as explained further below, the center lights D may be routed through a mirror box T to route light beams from the center lights D into the subject's eyes along a centerline. As another example, the cross lights B may provide a visual target for the subject during imaging to assist the subject with focusing on a centerline of the optical system R and the camera A for improved imaging.

The optical apparatus 100 may include various other lights, such as temporal lights D, nasal lights F, top lights G, and bottom lights H. The temporal lights D may be configured to provide light from a distal position relative to the subject's J eyes. For example, in certain configurations, the temporal lights E may be positioned between 15 and outward from a centerline of the respective left and right eyes. The nasal lights F may be configured to provide light from a proximal position relative to the subject's J eyes. For example, in certain configurations, the nasal lights F may be positioned between 15 and 45° inward from a centerline of the respective left and right eyes. In such configurations, nasal lights F may be used to illuminate opposing eyes. For example, in FIG. 1A-B, the lower nasal light F may be used illuminate the right eye of the subject J and the upper nasal lights F may be used to illuminate the left eye of the subject J. The top lights G may be positioned to provide illumination from above the subject J. In particular, each of the top lights G may be positioned between 15 and 45° above the subject's J eyes. Angles for the lights E, F, G, H may be adjustable. For example, the lights E, F, G, H may be detachable from the optical apparatus 100 for mounting at a different angle. In certain implementations, the optical apparatus 100 may including mounting points situated at regular intervals (e.g., every 5°, every 10°) to allow the lights E, F, G, H to be adjusted. The lights B, D, E, F, G, H may be implemented using one or more lighting apparatuses, such as LED lights. In certain implementations, the lights may project light in the visual spectrum. In additional or alternative implementations, the lights may project light at least partially outside of the visual spectrum (e.g., near infrared light). In certain implementations, near infrared light may be preferable, as such light may be less likely to damage the subject's J eyes and/or may allow the subject J to keep their eyes open for longer.

Light beams C from the lighting devices are depicted in FIGS. 1A-B, 2C-D, 3E, 9A-D, and 10A-D. Light beams C from certain lights may proceed directly from the lights to the eyes of the subject J. For example, light from the temporal lights E, nasal lights F, top lights G, and bottom lights H may proceed directly to the eyes of the subject J, with no intervening lighting components.

Additionally or alternatively, light beams from certain lights may be routed through various lighting components. For example, FIGS. 8A-D depict an interior of the mirror box T and FIGS. 9A-D depict the path of light through the mirror box T. The mirror box T includes mirrors N, beam splitters O, and plates P. The mirrors N may direct light from the center lights D (e.g., as depicted by the light segments U) towards the beam splitters O. The beam splitters O may be beam splitter mirrors configured to redirect a portion of the light from the mirror N towards the subject J and to allow the remainder of the light to pass through towards the plates P (e.g., as depicted by the light segments V). For example, the beam splitters O may be implemented as 70:30 beam splitter mirrors configured to redirect 70% of the light from the mirror capital letter N towards the subject J and to allow 30% of the light to pass through to the plates P, although other configurations may be used. To ensure proper lighting and imaging, it may be necessary for the light that passes through the beam splitters O to be absorbed by the plates P. For example, the light may be absorbed by an antireflective coating applied to the plates P. The portion of the light redirected towards the subject (e.g., depicted as light beam C in FIGS. 9C-D) may then be used to illuminate the eyes of the subject J for imaging.

As another example, light from the cross lights B may be redirected, as shown in FIGS. 10C-D. In particular, the optical apparatus 100 includes a beam splitter mirror S positioned in front of the camera sensor A and lenses R1-5 of the optical lens system R. The beam splitter mirror S redirects a portion of light received from the cross lights B towards the subject J. For example, the beam splitter mirror S may be a 70:30 beam splitter. The redirected light passes through lenses R6-7 of the optical lens system R and through the mirror box T to the subject J. In particular, the light beam C redirected by the beam splitter S may pass through the beam splitter O towards the subject J. In this way, both the cross lights B and the center lights D may be able to separately provide coaxial light towards the eyes of the subject J.

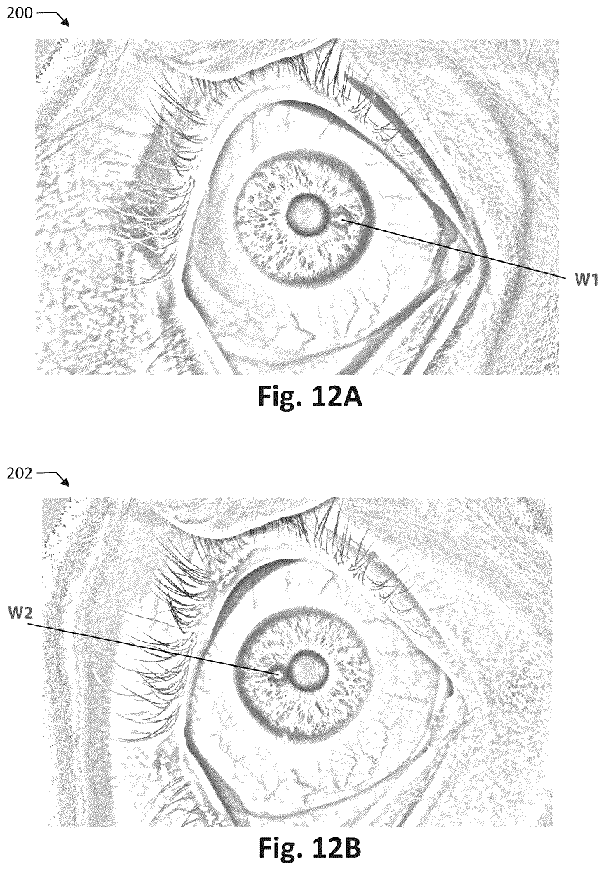

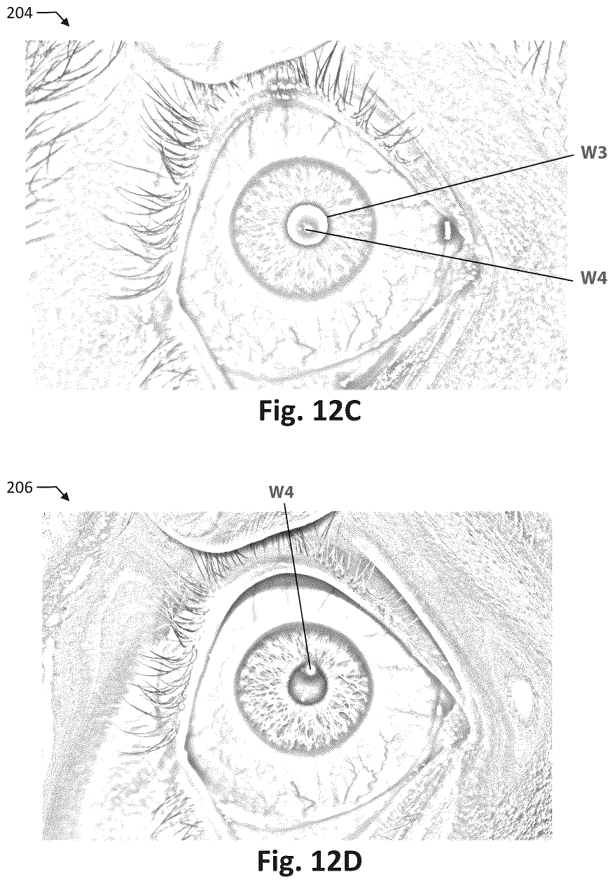

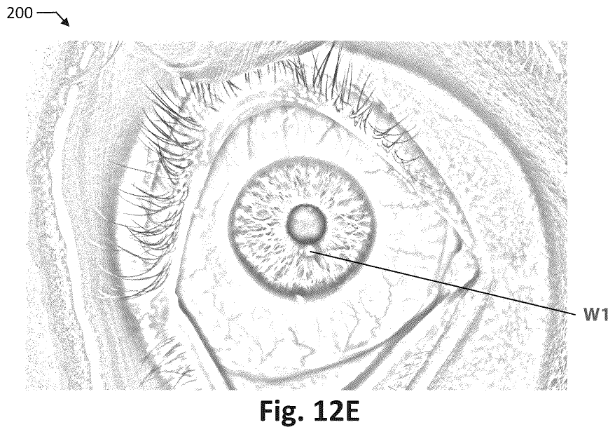

As explained above, the optical apparatus may be used to capture images of the subject's J eyes using the camera sensors A. FIGS. 12A-E depict exemplary images captured using an optical apparatus 100. In particular, FIG. 12A depicts an image 200 of a subject's J right eye illuminated using nasal lights F. FIG. 12B depicts an image 202 of a subject's J right eye illuminated using temporal lights E. FIG. 12C depicts an image 204 of subject's J a right eye illuminated using a coaxial light source (e.g., cross lights B and/or center lights D). FIG. 12D depicts an image 206 of a subject's J right eye illuminated using a top light G. FIG. 12E depicts an image 208 of a subject's J right eye illuminated using a bottom light H.

Each of these images 200, 202, 204, 206, 208 may provide unique information regarding the surface color and/or texture for various locations across a subject's J eye. For example, lights illuminating an eye from different angles may interfere differently with different layers of the eye. In the depicted images 200, 202, 204, 206, 208, the differences in interference may be seen by comparing the lights artifacts W1-6 that differ between the images 200, 202, 204, 206, 208. The image 200 includes a light artifact W1 positioned to the right of the pupil. Because the image 200 was illuminated using a nasal light F, the image includes brighter illumination for proximal portions of the eye located closer to the nose. The image 202 includes a light artifacts W to the left of the pupil. Because the image 202 was illuminated using a temporal light E, the image 202 includes brighter illumination for distal portions of the eye located further from the nose. The image 204 includes a light artifact W3 representing a reflection from an interior portion of the eye (e.g., the retina). The image 204 also includes a light artifact W4 located near the center of the pupil. Because the image 204 was illuminated using a coaxial light, the eye is more evenly lit within the image 204. Furthermore, coaxial light may be better able to penetrate one or more layers of the surface of the eye, allowing for an improved imaging of lower layers. The image 206 includes a light artifact W5 located near the top of the pupil. Because the image 206 was illuminated using a top light G, the image 206 may include brighter illumination for top portions of the eye. The image 208 includes a light artifact W6 near the bottom of the pupil. Because the image 208 was illuminated using a bottom light H, the image 208 may include brighter illumination for lower portions of the eye. Various techniques may be used to combine the information regarding various aspects and layers of the surface of an eye as depicted in the images captured by the optical apparatus 100.

In operation, the optical apparatus 100 may be configured to automatically take a predefined series of images of a subject's J eyes. For example, the optical apparatus 100 may first capture a first image of the eye illuminated by a coaxial light source (e.g., a center light D). The optical apparatus 100 may then capture a second image of the eye illuminated from a first side (e.g., using a nasal light F and/or a temporal light D). A third image may then be captured the eye illuminated from a second side (e.g., using a nasal light F and/or a temporal lights D). The second side may be opposite the first side. For example, the first side of the eye may be left and the second side of the eye may be right, or vice-versa. A fourth image may then be captured of the eye illuminated by a coaxial light source (e.g., the same coaxial light source used to illuminate the first image). A fifth image may then be captured of the eye when illuminated from above (e.g., using top lights G). A sixth image may then be captured of the eye when illuminated from below (e.g., using bottom lights H). A seventh image may then be captured of the eye when illuminated by a coaxial light source (e.g., the same coaxial light source used to illuminate the first image). In certain instances, when a non-coaxial light source is used (e.g., for the second, third, fifth, and sixth images), the cross light B may be illuminated to give the subject a visual target to focus on during imaging. In further implementations, the cross light B may be illuminated during the entire imaging process.

In practice, images may be captured of both of the subject's J eyes at the same time. For example, a first image of the left eye may be captured using a coaxial light source at the same time that a first image of the right eye is captured using a coaxial light source. When capturing images illuminated from the first and second sides, different types of light sources may be used for the left eye and the right eye. For example, when capturing a second image illuminated from the left side, a temporal light E may be used to illuminate the left eye and a nasal light F may be used to illuminate the right eye. As another example, when capturing the third image illuminated from the right side, a nasal light F may be used to illuminate the left eye and a temporal light E may be used to illuminate the right eye. Furthermore, in practice, the order of the first through seventh images may differ from that described above. For example, the sixth image may be captured before the fifth image. As another example, the fifth and sixth images may be captured before the second and third images. The additional images of the eye illuminated by a coaxial source (e.g., the fourth and seventh images) may be used to correct for slight movements of the subject J and/or the subject's J eye. In certain implementations, one or both of the fourth and seven images may be omitted. Additionally or alternatively, further coaxial images may be captured (e.g., between the second and third images and/or between the fifth and sixth images). Additional combinations or orderings of images and illuminations may be readily apparent to one skilled in the art in light of the present disclosure. All such combinations or orderings are hereby considered within the scope of the present disclosure.

In operation, the optical apparatus 100 may collect certain measurements regarding how the images of the subject's eyes were captured. For example, the optical apparatus 100 may collect position coordinates for the cameras A, such as vertical, lateral, and/or depth coordinates for the cameras A (e.g., relative to a particular “origin” position). A distance between each light B, D, E, F, G, H and the corresponding eye may be measured. Furthermore, an angle for certain lights (e.g., the lights E, F, G, H) may be measured relative to the axis of the camera A. For example, vertical angles may be measured between axis of the lights G, H and the axis of the camera A. As another example, horizontal angles may be measured between the axis of the lights E, F and the axis of the camera A. In certain instances, the angles may be controlled while imaging. For example, the optical apparatus 100 may include one or more alignment motors for one or more of the lights B, D, E, F, G, H. In such implementations, the angles may be captured by the alignment motors. Additionally or alternatively, the angles may be captured by angle sensors included in one or more of the lights B, D, E, F, G, H.

In still further implementations, an intensity of the lights B, D, E, F, G, H may be measured for various images captured by the optical apparatus 100. For example, the intensity may be measured in lumens, as a percentage of a maximum brightness, and/or as an amount of power used by the lights B, D, E, F, G, H. In certain implementations, the intensity of the lights B, D, E, F, G, H may be controlled from image to image. The intensity of the lights B, D, E, F, G, H may also be captured by one or more sensors (e.g., light sensors, power sensors, current sensors, voltage sensors). Such captured information may then be provided to software or other systems for analyzing the images (e.g., to create a 3-D model of all or part of the subject's eyes).

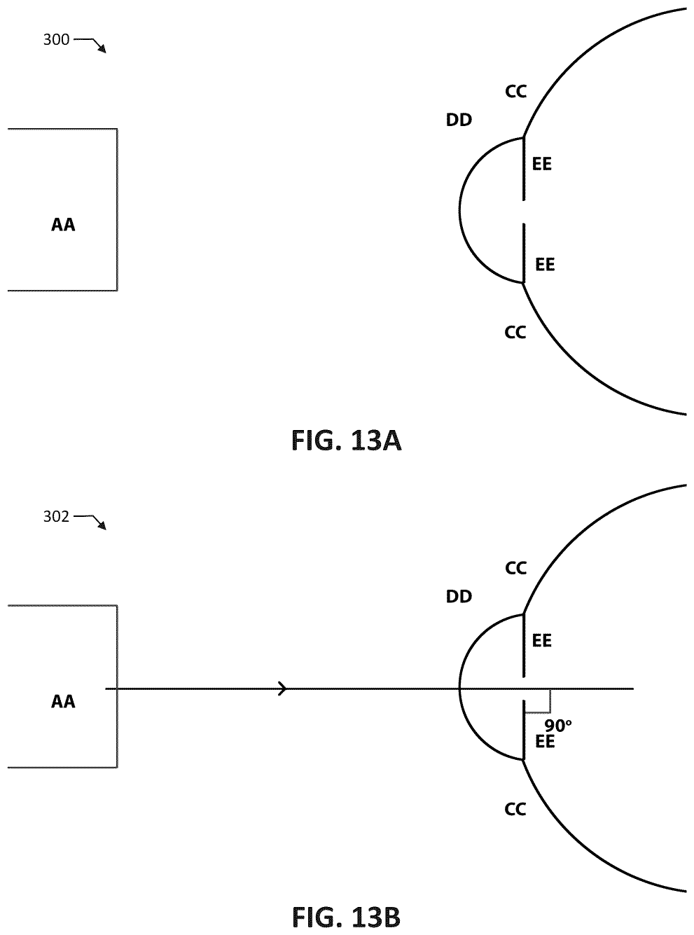

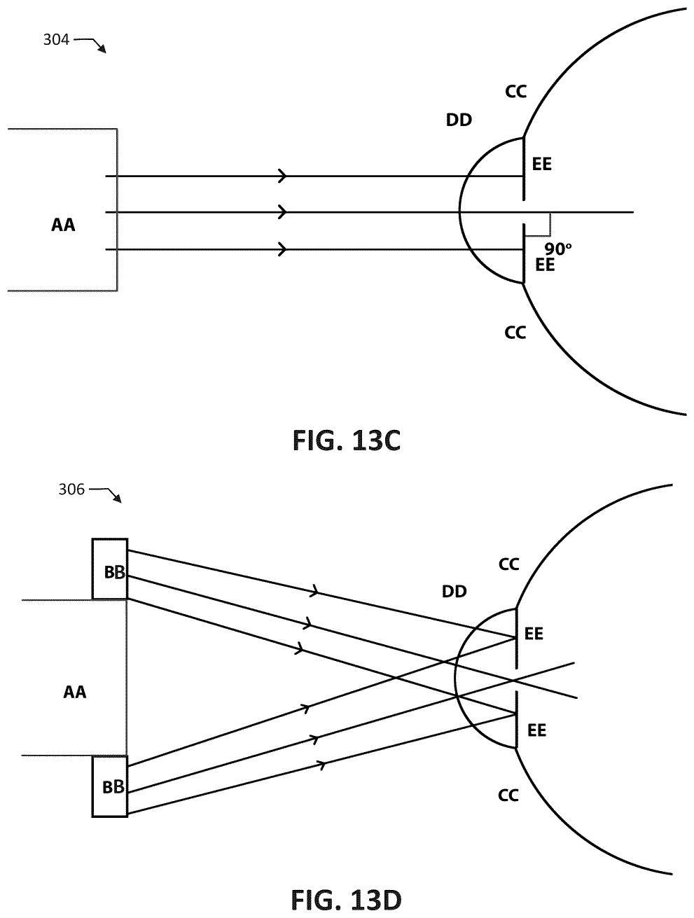

The above-discussed lighting systems and techniques may also result in improved images of a subject's eyes. In particular, FIGS. 13A-D depict lighting scenarios 300, 302, 304, 306 for imaging a subject's eye according to exemplary embodiments of the present disclosure. The lighting scenarios 300, 302, 304, 306 or merely illustrative and are accordingly not depicted to scale.

Beginning with FIG. 13A, the lighting scenarios 300 depicts an eye of a subject being imaged. The lighting scenario 300 includes a lens AA, the sclera CC of the eye, a cornea DD of the eye, and an iris plane EE of the eye. Turning to FIG. 13B, in the lighting scenario 302, the lens AA outputs a coaxial fixation light source that is coaxial to a center of the eye. The coaxial fixation light source may be an exemplary implementation of the fixation lights B. The narrow beam of the coaxial fixation light source requires the coaxial light source to align with an iris of the eye for the subject to see the light source, allowing the subject to align the iris plane EE perpendicular or nearly perpendicular with the coaxial light source.

In FIG. 13C, in the lighting scenario 304, the lens AA outputs a wider coaxial illumination light source emitting coaxial light towards the eye. The coaxial illumination light source emitted by the lens AA may be an exemplary implementation of the coaxial lights D. The eye may be aligned such that the iris plane EE is perpendicular or nearly perpendicular to the coaxial illumination light source. For example, the subject may be able to align the eye to the coaxial illumination light source using a coaxial fixation light, such as the light source depicted in the lighting scenario 300. Because the iris plane EE is perpendicular or nearly perpendicular, the iris plane EE is located closer to the light source and the lens AA and at a more consistent distance from the lens AA (e.g., because part of the iris plane EE is not angled away from the lens AA). Furthermore, because the iris plane EE is perpendicular, the iris plane EE may directly reflect back towards the lens AA, allowing a camera A to more directly capture the visual characteristics of the coaxial illumination light source.

In FIG. 13D, the lighting scenario 306 replaces the coaxial illumination light source with a ring light BB. The ring light BB may surround the lens AA and output light towards the eye. However, because the ring light surrounds the lens AA, the iris plane EE is not perpendicular to light received from the ring light BB. This increases the distance between the ring light BB and the Iris plane EE. Furthermore, less of the light received from the ring light BB may be reflected back towards the lens AA for use in imaging the eye and the iris plane EE.

Accordingly, as also discussed above, ring lights BB used in this manner may increase the number of visual and color artifacts and distortions present in images captured using ring light sources. As can be seen by comparing the lighting scenarios 304, 306, coaxial illumination light sources reduce these visual and clear artifacts by aligning the iris plane EE perpendicularly with the illuminating light source. Furthermore, the use of fixation light sources, such as that depicted in the lighting scenario 302, may improve the perpendicular alignment of the iris plan EE with the illuminating light source. In this way, the lighting systems used by the optical apparatus 100 may improve the quality of images captured by aligning a subject's eye properly with cameras A during imaging and by providing coaxial illumination of the eyes during imaging.

In practice, the optical apparatus 100 may be operated by a computing device. For example, the optical apparatus 100 may be operated by a computing device containing one or more computer programs or components. These components may be provided as a series of computer instructions on any conventional computer readable medium or machine readable medium, including volatile and non-volatile memory, such as RAM, ROM, flash memory, magnetic or optical disks, optical memory, or other storage media. The instructions may be provided as software or firmware, and may be implemented in whole or in part in hardware components such as ASICs, FPGAs, DSPs, or any other similar devices. The instructions may be configured to be executed by one or more processors, which when executing the series of computer instructions, performs or facilitates the performance of all or part of the operations of the optical apparatus 100.

It should be understood that various changes and modifications to the examples described here will be apparent to those skilled in the art. Such changes and modifications can be made without departing from the spirit and scope of the present subject matter and without diminishing its intended advantages. It is therefore intended that such changes and modifications be covered by the appended claims.

1. An optical imaging apparatus comprising:
a plurality of eye imaging systems, each eye imaging system comprising:
a camera sensor with a viewing direction oriented towards a subject whose eyes are imaged;
at least one coaxial light source, wherein the coaxial light source is configured to provide light coaxially aligned with the viewing direction of the camera sensor; and
a plurality of supplementary light sources configured to light an eye from different angles.

2. The optical imaging apparatus of claim 1, wherein the at least one coaxial light source includes a cross light to provide a fixation target for the subject during imaging.

3. The optical imaging apparatus of claim 2, wherein the cross light is oriented perpendicular to the viewing direction of the camera sensor.

4. The optical imaging apparatus of claim 3, wherein light from the cross light is redirected along the viewing direction by a beam splitter.

5. The optical imaging apparatus of claim 1, wherein the at least one coaxial light source includes a center light to illuminate the eye during imaging.

6. The optical imaging apparatus of claim 1, wherein light from the center light is redirected along the viewing direction through a mirror box including at least one mirror and at least one beam splitter.

7. The optical imaging apparatus of claim 1, wherein the at least one coaxial light source includes a cross light and a center light.

8. The optical imaging apparatus of claim 1, wherein the plurality of supplementary light sources includes at least two of: (i) temporal lights angled distally away from a centerline of the eye, (ii) nasal lights angled proximally away from a centerline of the eye, (iii) top lights angled above the eye, and (iv) bottom lights angled below the eye.

9. The optical imaging apparatus of claim 1, wherein the optical imaging apparatus is configured to take a plurality of images using at least a subset of the plurality of supplementary light sources, wherein at least a subset of the plurality of images are captured by illuminating a single supplementary light source from among the subset of the plurality of supplementary light sources.

10. The optical imaging apparatus of claim 1, wherein the optical imaging apparatus can adjust and measure a light intensity of the at least one coaxial light source and the plurality of supplementary light sources.

11. The optical imaging apparatus of claim 1, wherein the at least one coaxial light source and the plurality of supplementary light sources can illuminate on a continuous basis and a strobe basis.

12. The optical imaging apparatus of claim 1, wherein the optical imaging apparatus can adjust and measure an angle of the supplementary light sources.

13. The optical imaging apparatus of claim 1, further comprising a chin rest and forehead restraint, and wherein the optical imaging apparatus can adjust and measure at least one of (i) a distance between the plurality of eye imaging systems and the chin rest and forehead restraint and (ii) a distance between the at least one coaxial light source and the chin rest and forehead restraint.

14. The optical imaging apparatus of claim 1, wherein the at least one coaxial light source and the plurality of supplementary light sources are configured to emit light in at least a portion of the visual electromagnetic spectrum.

15. The optical imaging apparatus of claim 14, wherein the at least one coaxial light source and the plurality of supplementary light sources are configured to emit light at least partially in the near infrared light spectrum.

16. The optical imaging apparatus of claim 14, wherein the camera sensor is configured to detect light in at least the portion of the visual electromagnetic spectrum emitted by the at least one coaxial light source and the plurality of supplementary light sources.

17. The optical imaging apparatus of claim 1, wherein the optical imaging apparatus is configured to vertically adjust a position of the eye imaging systems and to horizontally adjust a position of the eye imaging systems.

18. An eye imaging system comprising:
a camera sensor with a viewing direction oriented towards a subject whose eyes are imaged;
at least one coaxial light source, wherein the coaxial light source is configured to provide light coaxially aligned with the viewing direction of the camera sensor; and
a plurality of supplementary light sources configured to light an eye from different angles.

19. The eye imaging system of claim 18, wherein the at least one coaxial light source includes a cross light to provide a fixation target for the subject during imaging.

20. The eye imaging system of claim 19, wherein the cross light is oriented perpendicular to the viewing direction of the camera sensor.
